(19) United States
(12) Patent Application Publication
(10) Pub. No.: US 2026/0130803 A1
(43) Pub. Date: May 14, 2026

(54) INCONTINENCE DETECTION STRIP AND METHODS

(71) Applicant: Peter Wengert, Sammamish, WA (US)

(72) Inventor: Peter Wengert, Sammamish, WA (US)

(21) Appl. No.: 18/940,833

(22) Filed: Nov. 8, 2024

(51) Int. Cl. A61F 13/42; A61F 13/49; A61F 13/84

(52) U.S. Cl. CPC A61F 13/42; A61F 13/49; A61F 13/84; A61F 2013/424; A61F 2013/426; A61F 2013/8479

(57) ABSTRACT

An apparatus and method for detecting incontinence events of people with limited mobility, are immobile, and/or non-responsive. One or more detection strips are place in close proximity to the person via either direct placement or through a secondary apparatus such as a pad, diaper or the like. The detection strip has an indicator pad attached to a thin carrier strip. The indicator pad may come in contact with urine or bowel matter. At any time, a caregiver may pull a strip and check the indicator pad for signs of an incontinence event. The caregiver can remove a strip without disrupting the person, wasting energy, or compromising the persons'dignity.

### CROSS-REFERENCE TO RELATED APPLICATIONS

Not related to this application.

### FEDERALLY SPONSORED RESEARCH AND DEVELOPMENT

Not related to this application.

### BACKGROUND OF THE INVENTION

This invention relates to a novel detection strip and methods for detecting if a non-responsive or limited mobility person has urinated or had a bowel movement.

Patients who have limited or no mobility, or are limited or non-responsive, such as those in end-of-life care, comatose states, or with severe neurological impairments, require extensive care to meet their daily needs. These patients are typically limited in their communication or voluntary movements, leaving them dependent on caregivers for tasks ranging from feeding to hygiene management. One of the most critical aspects of care for these patients is maintaining proper hygiene, particularly in managing incontinence. Due to the patients'inability to control their bladder or bowel movements, they are often at risk of being in a soiled state for a period of time. This can lead to significant discomfort, health complications, and challenges in care.

Caregivers, whether family members or healthcare professionals, are tasked with the responsibility of ensuring that the patient's hygiene is regularly maintained. In the absence of communicative ability, numbness from the affected area, or physical feedback from the patient, the caregiver must frequently check whether the patient has defecated or soiled themselves. Typically, this involves manually rolling or repositioning the patient to inspect the bed linens or clothing. This manual process, while essential, is labor-intensive and can be physically taxing, particularly for long-term caregivers who provide constant care for bedridden individuals. This is even more cumbersome when the patient is deemed to have a two-person or assisted lift requirement.

Repositioning immobile patients is not only physically demanding for caregivers but can also pose risks to the patients themselves. Patients with fragile skin, bed sores, pressure ulcers, or other medical conditions can experience discomfort, pain, or further injury when being moved. For caregivers, repetitive lifting, rolling, or repositioning of a patient can lead to musculoskeletal strain and injury, especially when handling larger or heavier individuals. This issue is compounded in cases where care is provided in home settings, where caregivers may not have access to specialized equipment like hospital beds designed for patient repositioning.

Beyond the physical challenges, the timely detection of soiling is crucial for maintaining the health and well-being of immobile patients. Prolonged exposure to moisture from urine or feces can lead to skin breakdown, resulting in infections, pressure ulcers, and other complications such as dermatitis or fungal growth. These conditions not only cause discomfort to the patient but can also lead to serious medical issues that require additional treatment, potentially increasing the burden on caregivers and healthcare systems.

Diapers, while commonly used for managing incontinence in immobile patients, present significant challenges for both caregivers and patients. Putting on diapers can be a difficult and time-consuming process, especially when dealing with non-responsive or limited mobility patients who require careful handling to avoid discomfort or injury. Once applied, caregivers must frequently check the diapers to ensure they haven't become soiled, which involves regularly lifting or repositioning the patient—tasks that can be physically taxing and disruptive. If diapers are not checked and replaced often enough, moisture from urine or feces can remain in contact with the skin for extended periods, leading to rashes, irritation, and an increased risk of infections such as diaper dermatitis. These complications can cause considerable discomfort for the patient and lead to further health issues, making frequent diaper changes essential yet cumbersome.

Pads or chucks, while intended to absorb waste, can also be particularly disruptive to a patient's dignity and comfort. These products often require caregivers to frequently remove coverings and to reposition the patient to check for soiling. For patients who are bedridden for long periods, this constant uncovering can be a source of disrupting sleep patterns, unnecessary discomfort and indignity, reducing their overall quality of care. While pads and chucks serve a functional purpose, they do not offer a respectful or dignified approach to managing incontinence for patients who deserve to be treated with as much comfort and privacy as possible.

Electronic pads, diapers, and bed sheets, designed to electronically detect moisture and alert caregivers when a patient has soiled themselves, may seem like an innovative solution, but they come with significant drawbacks. One of the primary concerns is their high cost, making them inaccessible for many long-term care facilities, home caregivers, and patients with limited resources. These devices also often require specialized equipment, regular maintenance, and technical support, adding complexity to what is already a demanding caregiving routine. Additionally, electronic solutions can be prone to malfunctions, false alarms, or battery failures, creating uncertainty and frustration for caregivers who rely on consistent and reliable performance. The complexity of integrating these systems into everyday care means they are far from a simple, practical solution for most caregivers, who often need more straightforward and cost-effective tools to manage incontinence effectively without adding financial and operational burdens.

The physical nature of detecting incontinence, combined with the associated risks to both patient and caregivers, underscores the need for an innovative and simple solution in the art of patient care. Current practices for monitoring and maintaining hygiene in people with limited or no mobility, or are non-responsive, are inadequate and complex for addressing the challenges faced in long-term care scenarios. Therefore, there is a significant demand for advancements in technology or care systems that can assist caregivers in identifying soiling incidents without the need for frequent disruptive and physically demanding manual checks. Such advancements would not only alleviate the physical burden on caregivers but also improve the quality of care for patients, reducing the likelihood of complications from prolonged exposure to unsanitary conditions.

### SUMMARY OF THE INVENTION

The present invention is an incontinence event detection strip it. In more detail, the present invention is a thin carrier strip with one or more indicator pads for use in checking if a person with limited or no mobility, or a non-responsive person, has had a urination or bowel movement event. The present invention provides a simple and low cost means of detecting incontinence events with minimal disruption to the non-responsive person. These and other features, aspects, and advantages of the present invention will become better understood with regard to the following description and accompanying drawings.

### BRIEF DESCRIPTION OF DRAWINGS

Preferred embodiments of the invention are described below with the reference to the following accompanying drawings:

FIG. 1 is a top view of the detection strip according to the present invention. The detection strip is shown with a carrier strip and indicator pad. A width of the carrier strip is shown.

FIG. 2 is a side view of the detection strip of FIG. 1. A thickness and a length of the carrier strip is shown.

FIG. 3 is a perspective view of a non-responsive or immobile person laying in a bed and on the detection trip of FIG. 1. A pull section of the detection strip is shown.

FIG. 4 is a perspective view of FIG. 3 and showing a caregiver pulling on the detection strip.

FIG. 5 is the perspective view of FIG. 4 with the caregiver inspecting the detection strip.

FIG. 6 is a partial perspective view of the indicator pad portion of the detection strip of FIG. 1 and showing the indicator pad in a non-soiled, or pristine, condition.

FIG. 7 is the perspective view of FIG. 6 with the indicator pad in a soiled condition.

FIG. 8 is a perspective view of a pad embodiment of the present invention and showing a plurality of the detection strip inserted into one or more openings.

FIG. 9 is a top view of the chucks pad of FIG. 8 and showing a plurality of openings without detection strips.

FIG. 10 is a perspective view of a non-responsive patient laying on the pad embodiment of FIG. 8.

FIG. 11 is a perspective view of a dual side pad embodiment of the pad embodiment of FIG. 8.

FIG. 12 is a top view of the dual side pad embodiment of FIG. 11 and showing multiple opening arrays.

FIG. 13 is the perspective view of the dual side pad embodiment of FIG. 11 and showing a detection strip removed. FIG. 13 includes cross-section arrow A-A.

FIG. 14 is a top view of a diaper embodiment of the present invention in an open state and showing a plurality detection strips of the present invention.

FIG. 15 is a perspective closed, or applied, embodiment of the diaper embodiment of the present invention shown in FIG. 14.

FIG. 16 is a top view of a non-square indicator pad alternative embodiment of the presentation invention.

FIG. 17 is a side view of a plurality indicator pad embodiment of the present invention.

FIG. 18 is a top view of a center indicator pad embodiment of the present invention.

FIG. 19 is a side view of a dual center indicator pad embodiment version of the present invention.

FIG. 20 is a top view of a handle alternative embodiment of the present invention having a pull handle on an opposite end of the indicator pad.

FIG. 21 is a partial section view along section arrow A-A of FIG. 13. Shown is carrier strip slidably mounted within an opening of the pad and in contract with a top surface and a bottom surface.

FIG. 22 is an alternative embodiment section view along section arrow A-A of FIG. 13. Shown is the carrier strip entering an opening and extending though a channel of the pad between the top surface and the bottom surface.

### DETAILED DESCRIPTION OF THE INVENTION

Many of the components and methods utilized in this invention are widely known and used in the field of the invention, and their exact nature or type is not necessary for a person of ordinary skill in the art or science to understand the invention; therefore, they will not be discussed in detail. Polymer, fabric and absorbent materials are well known in the art of medical and hygiene products and thus their exact composition does not need to be explained in significant detail for one to understand and practice the present invention without undue experimentation.

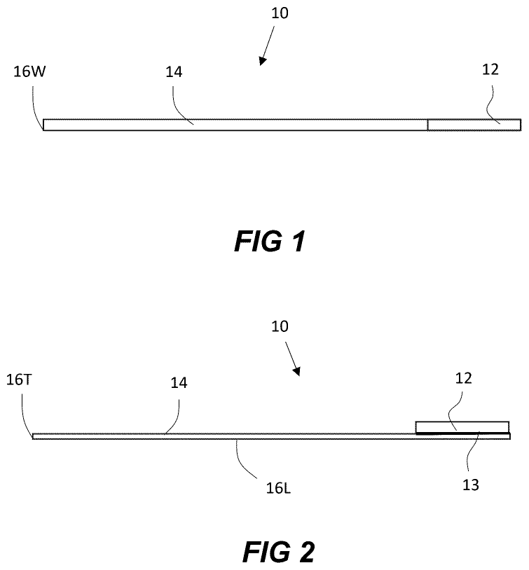

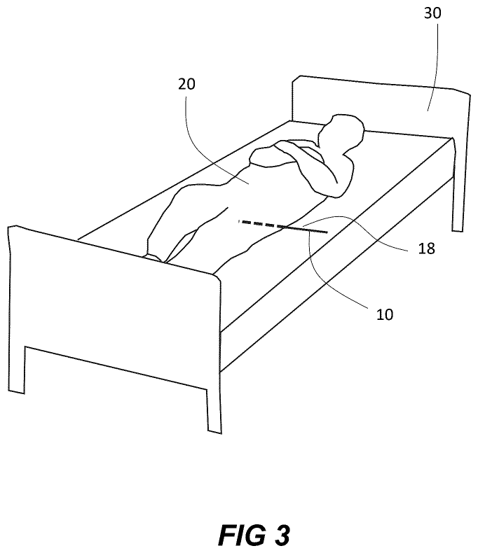

FIG. 1 and FIG. 2 show a detection strip 10 according to the present invention. Detection strip 10 is used by a caregiver 22 to determine if a non-responsive person 20 has had a bowel or urine event without having to disrupt person 20.

As used herein, the term “non-responsive” is used to describe person 20 which may be but is not limited to being actually non-responsive, as other applications may exist without departing from the spirt and scope of the present invention. For various reasons and according to the preferred embodiment of the present invention, “non-responsive” person 20 may be, but is not limited to, any one or combination, or of a similar state, of being unable to talk, be immobile, have limited mobility, have numbness, or be partially or completely incoherent. Such a state, or combination, may occur because of, but not limited to, neurological trauma, a coma, medications, and such. Person 20 may be in a bed 30 and in a resting or laying down position as shown in FIG. 3. While according to the best mode of the present invention, having person 20 being described as non-responsive and shown in bed 30, the present invention should not be construed to be limited to such a state. For any reason, it may be advantageous or necessary for caregiver 22 to want to check if person 20 has had a bowel or urination event without significant disruption to person 20, or without significant effort by caregiver 22. For further clarity, person 20 may be an infant.

In addition, person 20 may be, but is not limited to, being in a chair, hospital bed or standing. The present invention utilizes bed 30 as the best mode which may often occur when caring for an elderly family member by a family member care giver, but the present invention should not be construed to be limited to any situation or any type of apparatus or device that may be supporting person 20.

Also used herein, the term “plurality” is meant to indicate one or more of an object. In embodiments of the present invention, there may be the use of one or more of detection strip 10. A figure of the present invention may show a single instance of strip 10, or more. Each figure or described use of detection strip 10 may be optimal with any number of strip 10, including more or less than shown in any particular figure.

Detection strip 10 is comprised of one or more of an indicator pad 12 which is securely attached to a carrier strip 14. Carrier strip 14 is preferably made of a low friction plastic material that is biocompatible with the skin. Although not limited to any particular dimensions, the present invention has been successfully tested on adults using the material nylon, with a width 16W of around 0.25 inches, a thickness 16T less than 0.125 inches, a length 16L of 24 inches and a coefficient of friction against skin of less than 0.5. The purpose of carrier strip 14 is to place indicator pad 12 under person 20 in the region of likely urine or bowel matter of person 20 and enable caregiver 22 to pull the end opposite of indicator pad 12 and have carrier strip 14 and indictor pad 12 slide away from person 20. It should be appreciated that length 16L, width 16W, thickness 16T and material of carrier strip 14 can be optimized for specific patient sizes, weights and applications. Preferably carrier strip 14 is thin as to not cause skin or tissue issues for person 20. According to the preferred embodiment of the present invention, carrier strip 14 is flexible as to bend to the contour of person 20 in bed 30. While nylon has been described, carrier strip 14 may also be made from but is not limited to polymer materials such polyimide, polyethylene, and polypropylene which may have lower friction coefficients than nylon. Length 16L of carrier strip 14 may be any length optimal for a given application, but it has been found that 24 to 36 inches is sufficient to enable pad 12 to be optimally placed relative to an adult version of person 20 and have enough length to enable caregiver 22 to pull detection strip 10 without disrupting person 20. FIG. 3 shows a pull section 18 which is a portion of detection strip 10, and more particularly a portion of carrier strip 14, which is accessible by caregiver 22. In some applications, such as for infants or with use in diapers, the length of carrier strip may be 12 inches or less. The present invention should not be construed to be limited to any particular value of length 16L of carrier 14. While it has been found that 0.25 inches of width 16W is preferred, for some applications and embodiments 16W may be less than 0.25 inches and over 1 inch. While it has been found that 0.125 inches or less of thickness 16T is preferred, for some other applications and embodiments, such as using a slippery foam material for carrier 14, thickness 16T may be greater than 0.1 inches. Preferably, 16W and 16T and the material of carrier strip 14 is chosen to make strip 10 able to be pulled from under person 20, comfortable under person 20, strong enough not to break while being pulled, and wide enough to securely attach indicator pad 12.

Indicator pad 12 is preferably an absorbent biocompatible material that can capture an amount of surrounding bowel matter or urine, if such matter is present near person 20. It has been found the present invention will work with polymer materials common to diapers and chuck absorbent pads, such as cotton and polyester. Indicator pad 12 of the present invention should not be construed to be limited to any particular material, or combination of materials. Indicator pad 12 may be a porous foam.

Indicator pad 12 is attached to carrier strip 14 so that when carrier strip 14 is pulled by caregiver 22, indicator pad 12 moves with strip 14. According to the best mode of the presentation invention, indicator pad 12 is secured to strip 14 by an amount of adhesive 13, but the present invention should not be construed to be limited to such attachment method. Indicator pad 12 may be mechanically attached, and the like. Attachment methods are generally well known, and selecting an attachment method for a particular application or chosen materials can be determined without undue experimentation. Indicator pad 12 may also be removably attached to carrier strip 14 via hook-and-loop fasteners or the like as to enable a new indicator pad 12 to be attached to carrier strip 14 as warranted.

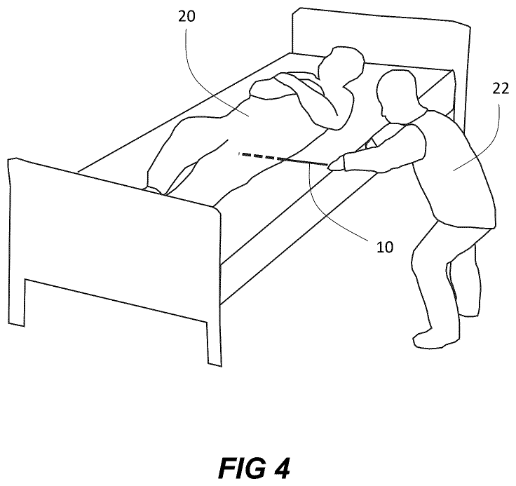

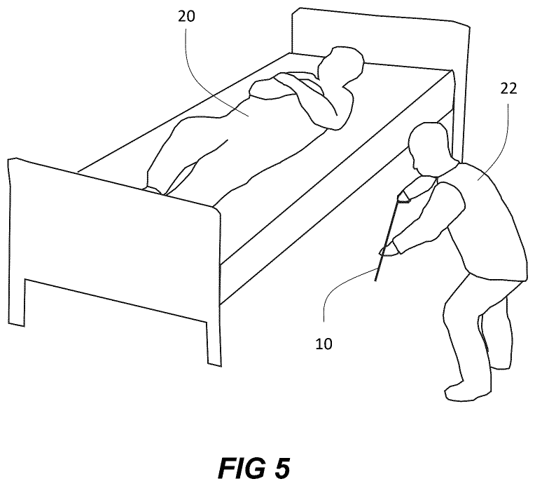

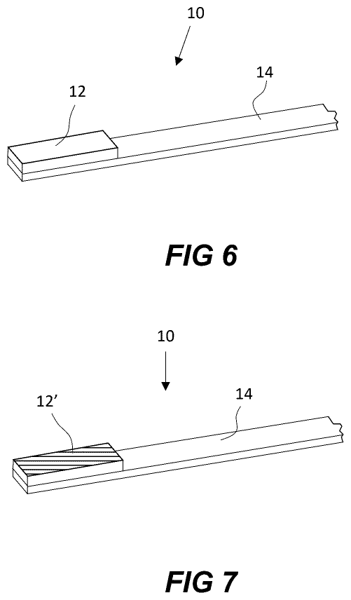

FIGS. 3, 4 and 5 show the general process of using detection strip 10. One or more of detection strip 10 is placed at least partially under person 20 with indicator pad 12 in proximity to a potential future amount of urine or bowel matter that may result from an incontinence event. The end of strip 10 opposite of indicator pad 12 is left accessible away from person 20 and is shown by pull section 18. Person 20 can rest, sleep and be comfortable on top of strip 10. As shown in FIG. 4, when an incontinence event is suspected, or after an amount of time has passed, caregiver 20 pulls the end of strip 10 causing strip 10 to slide relative to patient 20. As shown in FIG. 5, caregiver 22 then inspects indicator pad 12 for signs of urine or bowel movement. As shown in FIG. 6, indicator pad 12 may be in a pristine state indicating person 20 has not had an incontinence event. Such knowledge enables caregiver 22 to not unnecessarily disrupt person 20 or for caregiver 22 to needlessly expend energy or endure physical stress. Alternatively, as shown in FIG. 7, a soiled indicator pad 12′ may be in a discolored or wet soiled state due to the presence of urine or bowel matter. Soiled indicator 12′ provides caregiver 22 knowledge that person 20 has had an incontinence event and that person 20 should be attended to. Indicator 12 and its ability to capture incontinence matter provides the means to determine if person 20 is in a soiled state or not. While FIGS. 3, 4 and 5 show person 20 lying in bed 30 without any covers or blankets, it should be appreciated that the process described above and shown in the figures may be easily accomplished without uncovering person 20 if blankets or covers are used. The result of the present invention is that caregiver 20 may determine if person 20 has had an incontinence event without disrupting or removing the dignity of person 20 and without caregiver 20 spending significant energy moving person 20. Strip 10 also enables caregiver 22 to more quickly check person 20, enabling savings in time. In addition, and will be further appreciated by additionally described embodiments, a plurality of detection strip 10 may be placed under person 20. Whenever caregiver 22 decides to pull an instance of strip 10, if indicator pad 12 is in its pristine state, as shown in FIG. 6, caregiver can just wait and pull another instance of strip 10 in the future. The availability of multiple instances of strip 10 under person 20 and the ease at which they can be pulled, encourages caregiver 22 to more frequently check if person 20 has had an incontinence event. The result is that person 20 spends less time in a soiled state, thus reducing potential infections, skin irritations and unsanitary room conditions. It should be appreciated that particular conditions of person 20 may dictate any optimal number of strip 10 placed, including but not limited to one.

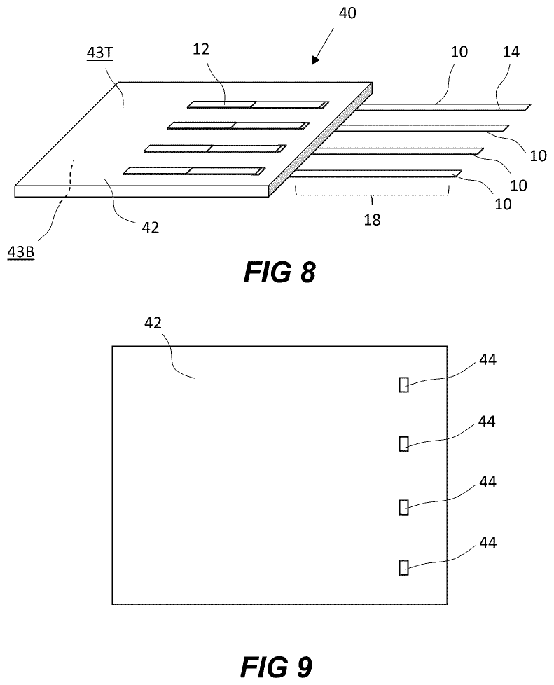

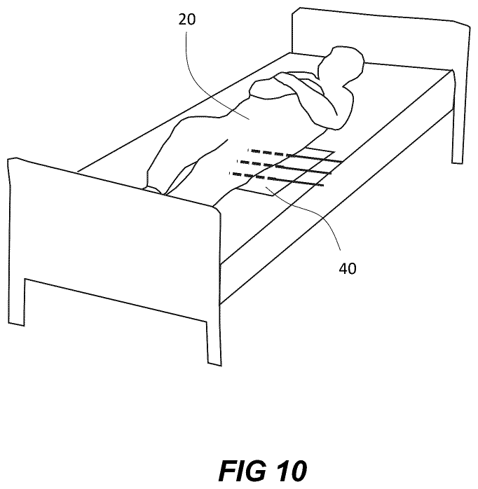

As shown in FIG. 8 and FIG. 9, the present invention may utilize chucks pads. Chucks pads are commonly used and well known in the field of medical care. They are placed under a patient and provide absorption of urine and bowel matter. When an incontinence event is found, the chucks pad can be removed from under the patient thus reducing risk of needing to replace bed sheets. FIG. 9 shows novel chuck assembly 40 according to the present invention. A chucks pad 42 may have one or more of an opening 44. As shown in FIG. 8, detection strip 10 may be inserted through opening 44 as to place indicator pad 12 along a top surface 43T of chucks pad 42 and in close proximity to person 20. The end of strip 10 opposite to indictor pad 12 is fed towards a bottom surface 43B of chucks pad 42 as to be accessible by caregiver 22 either through chucks pad 42 or to its bottom surface 43B nearest bed 30. For further clarity and as shown in FIG. 21, carrier strip 14 is fed through opening 44 and in contact with top surface 43T and below bottom surface 43B. Alternatively, and as shown in FIG. 22, carrier strip 14 may travel along top surface 43T, into opening 44 and between top surface 43T and bottom surface 43B through an opening channel 44′. Opening 44 provides the means to hold detection strip 10 relative to pad 42, to enable one or more of strip 10 to be preloaded to chuck pad 42 prior to being placed under person 20, and to reduce friction and any potential irritation to person 20. FIG. 10 shows chuck assembly 40 placed under person 20 in a similar fashion as the step shown by FIG. 3 and described above.

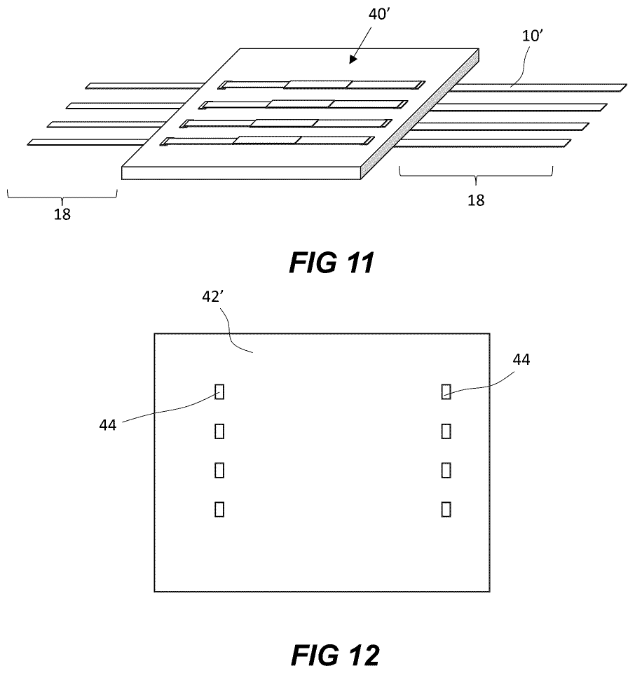

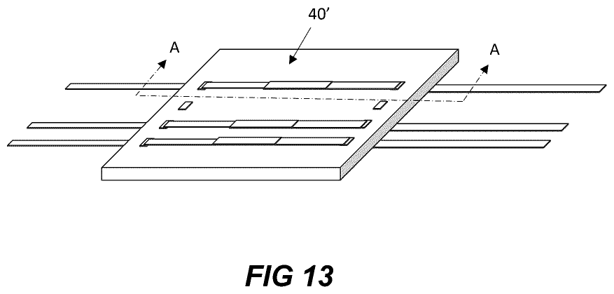

FIGS. 11 and 12 show an alternative embodiment of chucks assembly 40. Dual side chucks assembly 40′ has two arrays of openings 44 with each array on each side of a dual side chucks pad 42′. A dual side detection strip 10′ has approximately twice the length of detection strip 10 and a centrally attached indicator pad 12. Dual side detection strip 10′ is fed through opening 44 on each side of chucks pad 42′. The use of dual chucks assembly 40′ is the same as chucks assembly 40, but detection strip 10′ is more firmly secured to pad 42′ and caregiver 22 may pull either end of carrier 14 while standing on either side of bed 30. FIG. 13 shows one of the plurality of strip 10′ pulled and removed from dual side chucks assembly 40′.

While the chucks embodiment shown in the figures and described above is for a removable pad, it should be appreciated that the present invention is not limited to such a pad. The pad embodiment of the present invention is used to describe any object under person 20. Pad 42 should be understood to be, but not limited to, a bed sheet, a crib sheet, a blanked and the such, all withing the spirt and scope of the present invention.

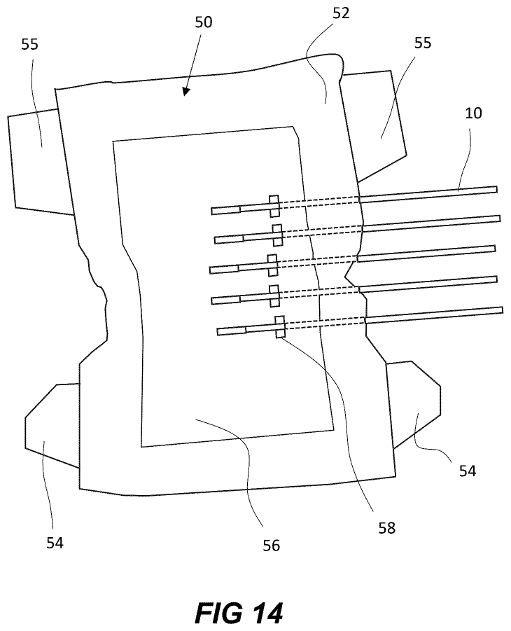

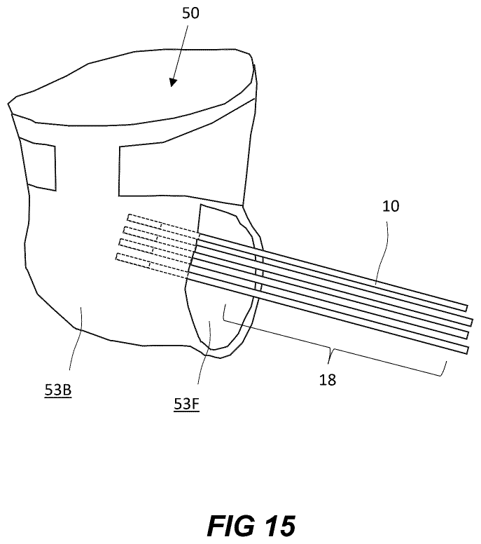

FIG. 14 shows a diaper embodiment of the present invention. Diapers are generally well known. Novel diaper assembly 50 is comprised of a diaper 52 having an absorbent pad 56, sealing flaps 55 and flaps 54. Novel diaper assembly 50 is applied to person 10 as is well known in the art. Novel diaper assembly 50 has an array of diaper openings 58, which similar to chuck assembly 40, enables one or more of detection strip 10 to have indicator pad 12 at an inside diaper surface 53F in close proximity to the skin of person 20 and have the opposite end of carrier strip 14 easily accessible by caregiver 22. Detection strip 10 may be fed between absorbent pad 56, between absorbent pad 56 and diaper 52, through diaper 52, or to an outside surface 53B of diaper 52. It has been found that transitioning strip 10 to outside surface 53B of diaper 52 provides acceptable results. FIG. 15 shows diaper assembly 50 in the as worn condition with a plurality of strip 10 ready for detection as described above for the chuck embodiment. One or more of detections strip 10 may be periodically pulled by caregiver 22 to detect if an incontinence event has occurred. Diaper assembly 50 may also be configured with dual side detection strip 10′ with similar motivations as dual side chuck assembly 40′. It should be appreciated that while the diaper embodiment described above is for a typical diaper containing absorbent pad 56, the preset invention should not be construed to be limited to such a typical diaper. The diaper embodiment of the present invention is intended to describe an article that attaches to person 20. As used herein and according to the present invention, a diaper may be any common article such as but not limited to underwear, a pull-on diaper, briefs, shorts, or medical device attached to person 20.

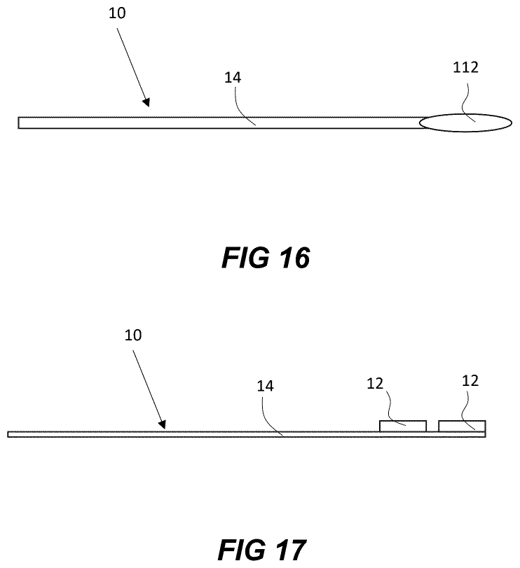

FIG. 16 shows an alternative indicator pad embodiment of the present invention. Attached to carrier strip 14 is an oval indicator pad 112. In some instances, oval pad 112 may be longer and wider than indicator pad 12. The present invention should not be construed to be limited to any size or shape of indicator pad 12.

FIG. 17 shows yet another alternative embodiment of detection strip 10. Attached to carrier strip 14 is plurality of indicator pad 12 in a spaced apart fashion. In some instances, conditions and body shapes of person 20 may make it advantageous to have a plurality of indicator pad 12. The present invention should not be construed to be limited to any number of indicator pad 12 mounted to carrier strip 14.

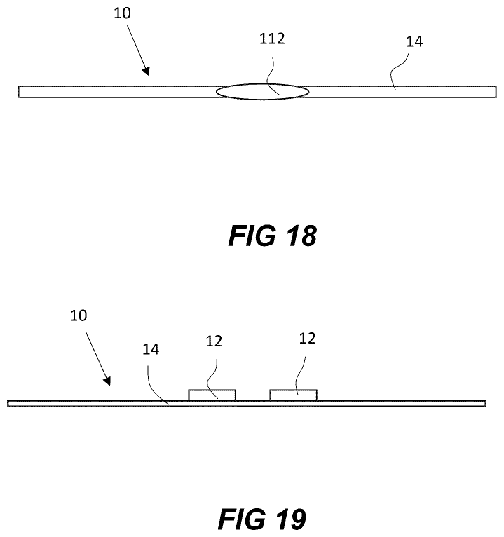

FIG. 18 and FIG. 19 show alternative location embodiments of the embodiments shown in FIGS. 16 and 17. In FIG. 18, oval indicator pad 112 is shown mounted in the center region of carrier strip 14. In FIG. 19 the plurality of indicator pad 12 are also mounted or attached centrally. Again, it should be appreciated that the present invention should not be construed to any particular location of indicator pad 12 relative to carrier strip 14.

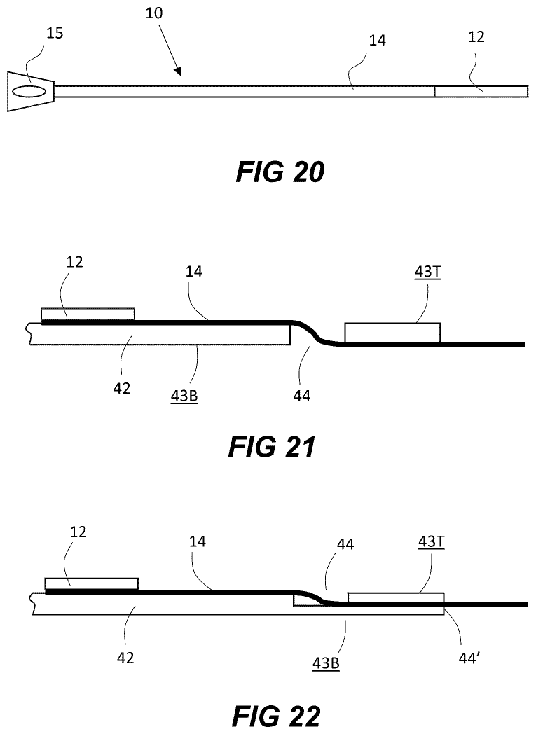

FIG. 20 shows yet another embodiment of the present invention. A handle 15 is formed into, or attached as a separate piece to, carrier strip 14. Handle 15 may extend outward to carrier strip 14 and optionally have a grasp slot to make it easier for caregiver 22 to remove strip 10 from underneath person 20. Handle 15 may be attached to both ends of carrier strip 14 for use in dual sided embodiments such as shown in FIG. 10.

While the detection strip system herein described constitute preferred embodiments of the invention, it is to be understood that the invention is not limited to these precise form of assemblies, process and methods, and that changes may be made therein without departing from the scope and spirit of the invention. Incontinence pads, bed sheets and clothing devices are all well known in the art and can leverage and benefit from the novel detection strip apparatus and methods disclosed herein.

I claim:

1. A detection strip comprising:
a carrier strip having a length, a width and a thickness, wherein said width is less than said length and said thicknesses is less than said width;
a pull section on a first end of said carrier strip;
at least one indicator pad mounted to a first side of said carrier strip and to a second end oppositive of said first end, and,
wherein said indicator pad transforms from a pristine state to a soiled state by the presence of incontinence matter from a non-responsive person.

2. The detection strip of claim 1 wherein said indictor pad is at least 0.25 inches in width.

3. The detection strip of claim 1, wherein said carrier strip has a coefficient of friction with skin less than 0.5.

4. The detection strip of claim 1, wherein said length of said carrier strip is at least 12 inches.

5. The detection strip of claim 1, wherein said detection strip is slidably mounted to a chucks pad.

6. The detection strip of claim 1, wherein said detection strip is slidably mounted to a diaper.

7. The detection strip of claim 1, wherein said first end includes a pull handle.

8. The detection strip of claim 1, wherein said carrier strip and said indicator pad are connected by an adhesive.

9. A pad assembly comprising:
an absorbent pad having a top surface, a bottom surface, and a plurality of openings going from said top surface and towards said back surface;
wherein each of said openings includes a slidably mounted detection strip having a carrier strip and at least one indicator pad;
said indictor pad is within said top surface of said absorbent pad and said indicator pad transforms from a pristine state to a soiled state in the presence of an amount of incontinence matter from a person; and,
wherein said carrier strip extends away from said absorbent pad to create a pull section.

10. The pad assembly of claim 9 wherein a caregiver may pull said pull section and remove said detection strip from said absorbent pad.

11. The pad assembly of claim 9 wherein said carrier strip has a length of at least 12 inches.

12. The pad assembly of claim 9 wherein said carrier strip has a coefficient of friction with skin less than 0.5.

13. A diaper assembly comprising:
a diaper having an inside surface, an outside surface, and a plurality of openings going from said inside surface towards said outside surface;
wherein each of said openings includes a slidable mounted detection strip having a carrier strip and at least one indicator pad;
wherein said indictor pad is within said inside surface of said diaper and capable of capturing an amount of incontinence matter from a person; and,
wherein said carrier strip extends away from said diaper to create a pull section.

14. The diaper assembly of claim 11 wherein a caregiver may pull said pull section and remove said detection strip from said diaper.

15. The diaper assembly of claim 11 wherein said carrier strip has a length of at least 12 inches.

16. The diaper assembly of claim 11 wherein said carrier strip has a coefficient of friction with skin less than 0.5.
